(19) United States
(12) Patent Application Publication
(10) Pub. No.: US 2004/0185561 A1
(43) Pub. Date: Sep. 23, 2004

(54) Animal product-free cell culture media extracts, supplements and culture media supplement systems

(75) Inventor: Paul Weldon Cook, Vancouver, WA (US)

Correspondence Address: KLARQUIST SPARKMAN, LLP, 121 SW SALMON STREET, SUITE 1600, PORTLAND, OR, 97204, US

(73) Assignee: Cascade Biologics, Inc.

(21) Appl. No.: 10/392,019

(22) Filed: Mar. 18, 2003

(51) Int. Cl. C12N 5/02

(52) U.S. Cl. 435/405

(57) ABSTRACT

Provided are APF plant extracts, APF plant extract cell culture media supplements, APF plant extract cell culture media, and APF plant extract cell culture media kits for the support of primary culture and cultivation of normal cells.

### FIELD

The present invention relates to cell culture media and cell culture media supplements.

### BACKGROUND

Basal cell culture media composed of purified salts, amino acids and vitamins are used to cultivate animal cells ex vivo. Typically these basal media are supplemented with animal-derived additives that can include fetal bovine serum (FBS), human serum, newborn calf serum, horse serum, and/or extracts from embryos, brain, pituitary gland and other tissues. Additionally, xenotrophic feeder layer cells are sometimes utilized to promote the primary culture of cells from animal tissues. These undefined animal-derived supplements and xenotrophic feeder layer cells are thought to act as important sources of growth factors, hormones, lipids, trace elements, vitamins, protease inhibitors, adhesion molecules, chelators and/or uncharacterized factors, which may act alone or in concert to promote cell survival, cell adhesion, and cellular proliferation.

Unfortunately, there are many disadvantages to the use of xenotrophic feeder layer cells, and animal-derived additives as cell culture supplements. These disadvantages may include, for example, contamination with infectious agents such as mycoplasma, viruses (e.g., HIV, Hepatitis) and prions (scrapie, bovine spongiform encephalopathy). Such infectious agents can pose a health risk to both workers exposed to the contaminated supplements and patients treated with therapeutic agents contaminated with infectious agents derived from the contaminated animal-derived supplements. Additionally, exposure of cultivated cells to infected animal-derived supplements can prove detrimental to the health and viability of cell cultures. The use of animal-derived supplements can also increase the cost of isolating and propagating both normal cells and immortalized cell lines derived from animal tissues. Thus, the development of animal product-free (APF) cell culture supplements may allow for increased safety to laboratory workers handling these reagents and/or foster the development of infectious agent-free cell cultures for production of therapeutic recombinant proteins, and the transplantation of cells and engineered tissue to humans.

Efforts have been made to eliminate animal-derived products from cell culture media. While serum has been eliminated from the culture medium used for some cell types, many of these media still contain animal-derived products such as extracts from embryos, brain, pituitary gland or other tissues, and recombinant growth factors derived from microorganisms cultured with animal-derived nutrients. Some media and media supplements that are APF have been described in the patent and commercial literature.

Use of extracts of aloe vera plant leaves has been suggested (see, for example, Published PCT Application No. PCT/US99/09574). Unfortunately, aloe vera plant leaf extracts do not possess the necessary efficacy to act as a replacement for animal-derived cell culture supplements for important cell types.

As shown in FIG. 1B, test results demonstrate that aloe vera leaf powder extracts do not possess activity sufficient to replace bovine pituitary extract (BPE) as a supplement; the extract lacks BPE's proliferation-promoting activity. The results shown in FIG. 1B were obtained as follows. Neonatal human epidermal keratinocyte “Target Cells” were thawed, and seeded at a density of 2.5×10³ viable cells/cm² in the wells of a 24-well plate into M2 Medium (i.e., EPILIFE® Medium (available from Cascade Biologics, Inc. of Portland, Oreg.) supplemented with “M2 Growth Factors”: hydrocortisone (Hyd, 30 nM), prostaglandin E-2 (PGE-2, 50 nM) (available from Sigma-Aldrich Chemical Co., St. Louis, Mo.), human recombinant insulin-like growth factor type-1 (IGF-1, 10 ng/ml) and human recombinant epidermal growth factor (EGF, 1 ng/ml)(available from PreproTech Inc., Rocky Hill, N.J.)). As used herein “Target Cells” refers to cells created by growing normal human neonatal keratinocytes (Cascade Biologics catalog number C-001-5C) in EPILIFE® Medium supplemented with EDGS (Cascade Biologics catalog number S-011-5) to the end of the second passage and cryopreserving the cells in suitable aliquots in a defined freezing medium (Synth-a-Freeze®, Cascade Biologics catalog number R-005) containing no growth factors.

Aloe vera whole leaf powder (obtained from Terry Laboratories of Melbourne, Fla., product no. WL004) was dissolved in a phosphate-buffered saline (PBS, pH 7.4 at a concentration of 60 g/L), sterile filtered through a 0.22 μM filter and added to the M2 Medium at the final concentrations indicated in FIG. 1B. As shown in FIG. 1A, positive and negative controls are represented by cultures grown in M2 Medium supplemented with 0.2% BPE or M2 Medium only, respectively. Proliferation of the cultures was assessed after 6 days of incubation at about 37° C., in a 5% CO₂/95% air atmosphere. Cells adhered to the culture surface were fixed in 70% isopropanol, stained with 0.1% methylene blue, excess stain removed by washing with water and the plates air dried. Processed cultures were set against a white background and images of each culture plate (FIG. 1A) were digitized using a flatbed scanner (Epson Perfection 1650). The optical density of each stained culture was determined from the digitized image using an image processing and analysis software program, such as IMAGEJ software (available on the internet at http://rsb.info.nih.gov/ij). The optical density of the stained cultures as determined by this procedure is representative of the number of cells in the culture wells and is, thus, one manner of measuring the efficacy of the materials in supporting the cultivation of the cells. All experimental points illustrated in FIG. 1B were performed in triplicate and each data point represents the average optical density relative to the negative control +/− standard deviation. Accordingly, aloe vera plant extracts were found not to possess the ability to support the primary culture (see Table 6 below) or cultivation of normal cells.

Other efforts have included APF supplements and media that utilize certain plant extracts as a replacement for animal products. Specifically, the uses of the hydrolysis products from wheat, rice, soybean, potato, and maize plants have been described. These plant extracts are hydrolyzed products primarily comprised of peptides derived from these plant proteins (see, e.g., U.S. Pat. No. 5,103,529, U.S. Pat. No. 5,741,705, and U.S. Pat. No. 5,885,835).

Currently available APF cell culture media supplement products include the HY-SOY (HY-SOY is a trademark of Sheffield Products Division of Quest International) and HYPEP products, soy and wheat-derived hydrolysates, respectively (available from Sigma-Aldrich Chemical Co., St. Louis, Mo.), AMPLICELL (available from BioMedia Europe), and APF supplemented media, such as CHO Protein-Free, Animal Component-Free Medium (available from Sigma-Aldrich Chemical Co., St. Louis, Mo.), “293 SFM II” and “VP-SFM” (available from Invitrogen Corporation, Carlsbad, Calif.). These APF supplements and media are used for the cultivation of immortalized established cell lines. Unfortunately, the cell lines must first be adapted to these APF cell culture environments prior to efficient long-term serial culture. Furthermore, as shown in Table 1 the HY-SOY and HYPEP supplements do not possess the necessary ability to support the cultivation and proliferation of normal cells.

Specifically, Table 1 shows results derived from testing several relevant concentrations (100-10000 mg/L) of HY-SOY and HYPEP 4601 plant-derived hydrolyzed cell culture supplements for their ability to support the growth of keratinocyte Target Cells. The data demonstrates that neither HY-SOY nor HYPEP 4601 hydrolyzed plant extract supplements possess activity such that these extracts could act as replacements for BPE to enhance proliferation of cultured human cells.

To achieve the results listed in Table 1, keratinocyte Target Cells were cultured in 24-well cell culture plates, and data was collected as described for FIGS. 1A and 1B. The plant-derived HY-SOY and HYPEP 4601 extracts (available from Sigma-Aldrich Chemical Company of St. Louis, product nos. 82522 and H6784, respectively) hydrolyzed cell culture supplements were dissolved in EPILIFE® basal medium at a concentration of 300 g/L, sterile-filtered through a 0.22 μM syringe filter and added to an M2 Medium at the final concentrations indicated in Table 1.

TABLE 1

| Supplement Added to M2 Medium | Relative Optical Density, +/−SD |
|---|---|
| No Addition | 0.0, +/−0.6 |
| BPE (0.2%) | 75.4, +/−3.4 |
| HY-SOY (100 mg/l) | 0.0, +/−0.5 |
| HY-SOY (300 mg/l) | 0.7, +/−0.2 |
| HY-SOY (1000 mg/l) | 1.9, +/−0.3 |
| HY-SOY (3000 mg/l) | 0.8, +/−0.6 |
| HY-SOY (10000 mg/l) | 0.0, +/−0.4 |
| HYPEP 4601 (100 mg/l) | 0.0, +/−0.4 |
| HYPEP 4601 (300 mg/l) | 0.0, +/−0.9 |
| HYPEP 4601 (1000 mg/l) | 2.1, +/−0.3 |
| HYPEP 4601 (3000 mg/l) | 0.0, +/−0.5 |
| HYPEP 4601 (10000 mg/l) | 0.0, +/−0.5 |

### SUMMARY

Provided are APF plant extracts, APF plant extract cell culture media supplements, APF plant extract cell culture media, and APF plant extract cell culture media kits that support the primary culture and cultivation of normal cells.

In one embodiment, a cell culture medium comprises a supplement comprising a non-hydrolyzed plant extract, wherein the culture medium supports the cultivation of cells from animal tissue, such as keratinocytes, mammary epithelial cells, and/or corneal cells, in vitro.

In another embodiment, a cell culture medium comprises a supplement comprising an extract from a portion of at least one seed-bearing plant, such portion comprising, for example, a dormant and/or germinated derivative of a seed, wherein the culture medium supports the cultivation of cells from animal tissue, such as keratinocytes, mammary epithelial cells, and/or corneal cells, in vitro.

In another embodiment, a cell culture medium comprises a supplement comprising a non-hydrolyzed extract from a portion of at least one seed-bearing plant, such portion comprising, for example, a dormant and/or germinated derivative of a seed, wherein the culture medium supports the cultivation of cells from animal tissue, such as keratinocytes, mammary epithelial cells, and/or corneal cells, in vitro.

In another embodiment, a cell culture medium having a supplement comprising a non-hydrolyzed plant extract derived from at least a portion of a storage organ of a seed-bearing plant, wherein the culture medium supports the primary culture and/or propagation of cells from animal tissue, such as keratinocytes, mammary epithelial cells, and/or corneal cells, ex vivo.

In other embodiments, the plant extracts are combined with a basal medium.

In one embodiment of a method for cultivating an animal cell, the method comprises contacting the animal cell, such as keratinocytes, mammary epithelial cells, and/or corneal cells, with an embodiment of the disclosed culture medium supplement and cultivating the animal cell under conditions suitable to support cultivation of the animal cell.

Further embodiments of the extracts, supplements, cell culture media, cell culture media kits, and methods are discussed below.

### BRIEF DESCRIPTION OF THE DRAWINGS

FIG. 1A shows images of untreated and BPE-treated cell culture controls.

FIG. 1B is a graph depicting cell culture responses to aloe vera leaf extract supplements at several concentrations (300-600 mg/L). As shown, the aloe vera extract has no proliferation activity for cultured normal human cells.

FIGS. 2A-2D are images showing keratinocyte cultures added to particular embodiments of russet potato plant tuber extract cell culture media and supplements as disclosed herein.

FIG. 3 is a graph demonstrating cell culture proliferation activity of particular embodiments of peanut extract supplements and media, wherein the peanut extract supplements and media comprise molecules and/or molecular complexes of average molecular mass predominantly above 100,000 Daltons.

FIGS. 4A and 4B are graphs depicting cell culture responses to various embodiments of peanut/potato extract supplements in various basal media.

FIGS. 5A and 5B depict various cell cultures and the cell cultures' responses to various embodiments of peanut/potato extract supplements and media.

FIGS. 6A and 6B depict various cell cultures and the cell cultures' responses to various embodiments of peanut/potato extract supplements and media.

### DETAILED DESCRIPTION

Disclosed are animal-product free (APF) extracts, supplements, cell culture media systems, and related methods. The extracts, cell culture media supplements, and cell culture media systems provide for the primary culture and/or subsequent cultivation of normal cells derived from animal tissue, e.g., keratinocytes, mammary epithelial cells and/or corneal epithelial cells. More specifically, disclosed are seed-bearing plant and/or non-hydrolyzed plant extracts, cell culture media supplements including such extracts, cell culture media systems including such extracts, and related methods.

The culture media supplements may be 1× supplements or may be concentrated. The culture media may comprise a number of ingredients, including amino acids, vitamins, organic and inorganic salts, sugars, etc., each in amounts that may support the cultivation of an animal cell in vitro. The culture media supplements and systems may be used to culture a variety of animal and human cells.

Unless otherwise indicated, all numbers expressing quantities of ingredients, properties such as molecular weight, reaction conditions, and so forth used in the specification and claims are to be understood as being modified by the term “about.” Accordingly, unless indicated to the contrary, the numerical parameters set forth are approximations that may depend upon the desired properties sought.

The terms “a,” “an,” and “the” are understood to include the plural of the object referred to, as well as the singular, unless the context indicates otherwise. This is true although the terms “one or more” or “at least one” may be used in the specification and/or claims. “Comprising” means “including.” Although methods and materials similar or equivalent to those described herein can be used in the practice or testing of the present disclosure, suitable methods and materials are described below. The materials, methods, and examples are illustrative only and not intended to be limiting.

Definitions

“Culture” or “cell culture” refers to the maintenance, growth and/or differentiation of cells in an in vitro environment. The term “cell culture,” however, is a generic term and may encompass the cultivation of individual cells, tissues, organs, organ systems, or whole organisms, for which the terms “tissue culture,” “organ culture,” “organ system culture” or “organotypic culture” may occasionally be used interchangeably with the term “cell culture.”

“Cell culture media,” “culture media” (singular “medium” in each case), “supplement” and “media supplement” refer to nutritive compositions that cultivate cell cultures.

“Cultivate” refers to the sustaining, propagating (growing) and/or differentiating of cells outside of tissue or the body, for example in a sterile plastic (or coated plastic) cell culture dish or flask. “Cultivation” may utilize a culture medium as a source of nutrients, hormones and/or other factors helpful to propagate and/or sustain the cells.

“Culture vessel” may refer to a glass, plastic, metal or other container that provides an environment for culturing cells.

“Cytokine” refers to a compound for inducing a physiological response in a cell, such as growth, differentiation, senescence, apoptosis, cytotoxicity or antibody secretion. “Cytokine” includes growth factors, interleukins, colony-stimulating factors, interferons, and lymphokines.

“Enzymatic digest” refers to a composition comprising a specialized type of extract, namely one prepared by treating the extracted material with one or more enzymes capable of breaking down the components of the extract into simpler forms.

“Extract” refers to a composition that may be formed by treatment of the material mechanically (e.g., blending, grinding, pressure treating) and/or chemically (e.g., by distillation, precipitation, high salt treatment) usually in water or aqueous buffer.

“Ingredient” refers to any compound or other material, whether chemical or biological in origin that may be used in cell culture media to maintain and/or promote the growth and/or differentiation of cells. The terms “component” “nutrient” and “ingredient” may be used interchangeably. Conventional ingredients used for cell culture media may include but are not limited to amino acids, salts, metals, sugars, lipids, nucleic acids, hormones, vitamins, fatty acids, proteins and the like. Other ingredients that promote and/or maintain cultivation of cells ex vivo may be selected by those persons of ordinary skill in the art as required for a desired affect.

“Differentiate” or Differentiating refers to a cell or cell culture producing particular gene products typically associated with a specific function or functions commonly associated with a particular tissue or tissues. Highly differentiated cells are generally not proliferative, while conversely, highly proliferative cells generally do not express particular gene products typically associated with differentiation.

“Proliferate” refers to the property of one cell dividing into two essentially identical cells or a population of cells increasing in number (e.g., to reproduce).

“Propagation” refers to growing (e.g., reproducing via cell proliferation) cells outside of tissue or the body, for example, in a sterile container such as a plastic (or coated plastic) cell culture dish or flask.

“Adhere” refers to cells attaching to a vessel, for example, a cell attaching to a sterile plastic (or coated plastic) cell culture dish or flask in the presence of an appropriate culture medium. Certain classes of cells are not sustained or do not grow in a culture unless they adhere to the cell culture vessel. Certain classes of cells (“non-adherent cells”) are maintained and/or proliferate in culture without adhering.

“Isolate” or “isolating” refers to separating and collecting individual cell cultures from tissue or the body.

“Primary culture” refers to cells, tissue and/or culture where the isolated cells are placed in a first culture vessel with culture medium. The cells, tissue and/or culture may be sustained and/or may proliferate, however, as long as the cells, tissue and/or culture remain in the first vessel the cells, tissue and/or culture are referred to as the primary culture.

“Capable of supporting the cultivation” refers to a condition that allows the survival and, in some cases, proliferation and/or differentiation, of isolated cells and/or tissues in culture. As is known to those of ordinary skill in the art, what survival and/or proliferation and/or differentiation rates are required to be considered supporting is dependent upon the use to which the culture will be put. For example, to assess the proliferative efficacy or toxicological properties of certain compounds a relatively small number of cells per sample (e.g., 10-10,000) may be required, whereas to utilize the cell cultures in a manufacturing process to obtain cell-derived and/or therapeutic products the desirable number of cells in a culture could be much higher, e.g., from 10⁶ to 10¹². In addition, culture requirements for initial stages of a primary culture of a particular cell type may be more numerous and stringent than subsequent requirements of the same cells once they have been cultured for a period of time.

“Long-term cultivation” refers to the cultivation of cells for long periods of time (months and/or years).

“Spread” refers to a cell extending itself after it adheres to a cell culture vessel.

“Plating” refers to a cell adhering to and spreading on a cell culture vessel.

“Serial passage” refers to the act of diluting and subdividing cells into multiple vessels when the cells have proliferated to a desired extent. As cells are passaged from the primary culture vessel into a subsequent set of vessels, the subsequent cultures may be referred to herein as “secondary culture” or “first passage,” etc.

“Normal cells” refer to cells that are considered by persons of ordinary skill in the art to be genetically normal and possess a finite life span when placed into cell culture. “Abnormal cells” (e.g., tumor cells, genetically modified cells) refer to cells that typically display unlimited life spans.

“Non-hydrolyzed” as used herein in reference to plant extracts means an extract of plant material that has not been treated with additional enzymes or chemicals in order to facilitate hydrolysis of proteins contained therein; or an extract of plant material that has been treated to facilitate hydrolysis but the treatment ceases prior to complete or substantially complete hydrolysis of proteins contained therein. A “non-hydrolyzed” extract may contain a minor amount of hydrolyzed proteins due to hydrolysis by endogenous enzymes.

“Seed” as used herein in reference to plants and/or plant materials means a plant propagating organ formed in the sexual reproductive cycle of seed-bearing plants (Spermatophyta), containing all or some of the following parts: a plant embryo, a food supply (e.g., endosperm), and a seed coat. Examples of seeds include but are not limited to peanuts, beans, sunflower seeds, hazelnuts, almonds, corn, and rice.

“Storage organ” as used herein in reference to plants and/or plant materials means a modified stem or root of a plant which acts as a nutritive storage reservoir and can support plant vegetative reproduction from associated nodes or axillary buds. Examples of plant storage organs include but are not limited to: roots, modified roots (e.g., carrots, sweet potatoes), modified stems (e.g., in potatoes, yams), runners (e.g., strawberries), rhizomes (e.g., bermuda grass), corms (e.g., gladiolus), tubers (e.g., potatoes, yams) and bulbs (e.g., onions).

“Seed-bearing” or “Seed” plants as used herein means those plants belonging to the group of plants referred to as Spermatophyta and commonly gymnosperms and angiosperms.

It is to be understood that although we may refer to only “cells” in particular instances in the specification, unless otherwise noted, the term “cells” includes individual cells, tissues, organs, organ systems, and/or whole organisms.

Basal Media

Basal media in the disclosed cell culture supplements and cell culture systems may be aqueous based and may comprise a number of ingredients in a solution. Any basal media may be used and may include, for example, one or more of the following: amino acids, vitamins, organic salts, inorganic salts, trace elements, buffering salts, sugars, ATP, and the like (suitable basal media ingredients are available from Sigma-Aldrich of Saint Louis, Mo.).

Such ingredients, when admixed in solution form a “basal medium.” Suitable commercially available basal media include any basal medium suitable to support the maintenance, growth, and/or differentiation of a desired cell type as is known to those persons of ordinary skill in the art, such as medium MCDB 153 (Catalog # M7403), medium F12 (Catalog # N6658), medium RPMI 1640 (Catalog # R8758), Dulbecco's Modified of Eagle's medium (DME, Catalog # D5796) available from Sigma-Aldrich, of St. Louis, Mo. Keratinocyte Basal Medium (KBM, Catalog # CC-3101), Mammary Epithelial Cell Basal Medium (MEBM, Catalog # CC-3152), all available from Cambrex BioScience Walkersville, Inc., Walkersville, Md. Medium 154 (Catalog # M-154) and EPILIFE® Medium (Catalog # M-EPI), both available from Cascade Biologics, Inc., Portland, Oreg. Similar media are available from other suppliers (e.g., Invitrogen Corporation, Carlsbad, Calif.).

For example, a suitable basal media may include the ingredients, in a solution, as shown in Table 2. The ingredients of such a basal medium would be present in concentrations suitable to support the maintenance, growth, and/or differentiation of a desired cell type as is known to those persons of ordinary skill in the art.

TABLE 2
Example Basal Medium Components

| | |
|---|---|
| L-Alanine | Adenine |
| L-Arginine.HCl | Choline Chloride |
| L-Asparagine.H₂O | D-Glucose |
| L-Aspartic Acid | myo-Inositol |
| L-Cysteine.HCl.H₂O | Putrescine.2HCl |
| L-Glutamic Acid | Sodium Acetate.3H₂O |
| L-Glutamine | Sodium Pyruvate |
| Glycine | Thymidine |
| L-Histidine.HCl.H₂O | CaCl₂.2H₂O |
| L-Isoleucine | KCl |
| L-Leucine | MgCl₂.6H₂O |
| L-Lysine.HCl | NaCl |
| L-Methionine | Na₂HPO₄.7H₂O |
| L-Phenylalanine | CuSO₄.5H₂O |
| L-Proline | FeSO₄.7H₂O |
| L-Serine | Na₂SeO₃ |
| L-Threonine | MnSO₄ |
| L-Tryptophan | Na₂SiO₃.9H₂O |
| L-Tyrosine | (NH₄)₆Mo₇O₂₄.4H₂O |
| L-Valine | NH₄VO₃ |
| d-Biotin | NiCl₂.6H₂O |
| Folic Acid | SnCl₂.2H₂O |
| DL-α-Lipoic Acid | ZnSO₄.7H₂O |
| Niacinamide | HEPES |
| D-Pantothenic Acid | NaOH |
| Pyridoxine.HCl | NaHCO₃ |
| Riboflavin | Phenol Red (Na salt) |
| Thiamine.HCl | Vitamin B12 |

An alternative suitable basal medium may include ethanolamine and O-phosphorylethanolamine in addition to those ingredients listed in Table 2.

Cell Culture Media Supplements

Cell culture supplements typically include serums, extracts, growth factors, hormones, cytokines and the like. The cell culture media supplements disclosed herein include one or more non-hydrolyzed plant extracts and/or extracts derived from a portion of one or more seed-bearing plants, enzymatic digests, and optionally purified peptides, proteins and hormones. The cell culture media supplements may comprise extracts derived from plants treated by dialysis wherein the retentant from the treatment of the extracts are predominately greater than 100 Kd in mass.

Cytokines used in the culture media may include, for example, one or more of the following: growth factors such as epidermal growth factor (EGF), acidic fibroblast growth factor (aFGF), basic fibroblast growth factor (bFGF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor beta (TGF-β3), vascular endothelial cell growth factor (VEGF) transferrin, various interleukins (such as IL-1 through IL-18), various colony-stimulating factors (such as granulocyte/macrophage colony-stimulating factor (GM-CSF)), various interferons (such as IFN-γ) and other cytokines having effects upon hematopoietic stem cells such as stem cell factor (SCF) and erythropoietin (Epo). These cytokines may be obtained commercially, for example from R&D Systems, Minneapolis, Minn., and may be either natural or recombinant. Most preferably, for culture of a wide variety of mammalian cells, the basal media will contain EGF at a concentration of about 0.01-100 ng/ml, preferably about 0.1-10 ng/ml, and most preferably about 0.5-5 ng/ml. Other cytokines, if used, may be added at concentrations that are determined empirically or as guided by the established cytokine art.

Additional ingredients that may be included in the present media are insulin (especially as insulin Zn⁺⁺) and transferrin. These additional ingredients, available commercially (for example, from Sigma-Aldrich, St. Louis, Mo.), may be formulated into the present media at concentration ranges of about 0.1 to about 100 μg/ml or about 1 to about 10 μg/ml. Additionally, recombinant insulin or zinc based salt of insulin may be substituted for animal- or human-derived insulin. Other ingredients or substitutes may be added to the supplement compositions as are known to those persons of ordinary skill in the art.

Cytokines and like components of the supplements may instead (or in addition) be included in the basal media. Such components are typically included with the supplement compositions as the supplement compositions are conventionally stored at about −20° C. rather than the about 4° C. temperature regularly used for storing basal media. Cytokines and like components may fair better at temperatures closer to −20° C.

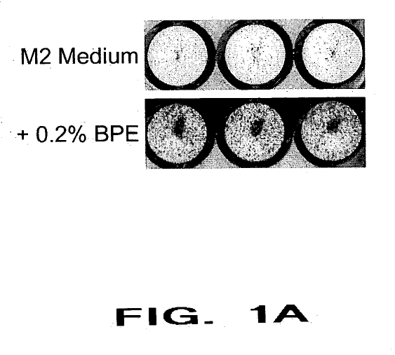

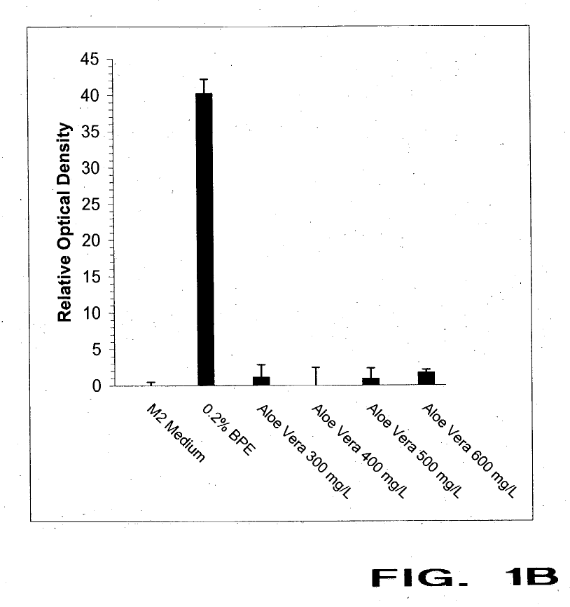

Plants and plant materials suitable for the disclosed culture supplements and media include but are not limited to seed-bearing plants, developing, dormant or germinated seeds, such as sunflower—Helianthus annuus L., pumpkin—Cucurbita pepo L., alfalfa—Medicago sativa L., poppies—Papaver Somniferum, flax—Linum usitatissimum, corn—Zea mays L., millet—Panicum miliaceum L., Quinoa—Chenopodium quinoa Willd., sesame—Sesamum Indicum, almonds—P. amygdalus Batsch, hazelnuts—Corylus avellana L., brazil nuts—Bertholletia excelsa Humb., cashews—Anacardium occidentale L., pine nuts—Pinus pinea L., pecans—Carya illinoinensis, walnuts—Juglans californica S. Wats., filbert nuts—Corylus maxima Mill, beans—Phaseolus vulgaris L., peanuts (Spanish and Virginia peanuts)—Arachis hypogaea L., lentils—Lens culinaris Medik, peas—Pisum sativum L., chick peas—Cicer arietinum L., soybeans—Glycine max (L.) Merrill, barely—Hordeum vulgare L., rice—Oryza. sativa L., wheat—Triticum aestivum, Triticum sativum; and/or storage organs including roots, modified roots, modified stems, tubers, rhizomes, corms, runners and bulbs, for example, potato-related tubers such as potatoes (e.g., russet, white, red, yellow)—Solanum tuberosum L, yams—Dioscorea alata L., sweet potatoes—Ipomoea batatas (L.) Poir, carrots—Daucus carota L., and turnips—Brassica rapa L. Aloe vera plant leaf extracts are specifically excluded from the group of plant-derived extracts disclosed herein as aloe vera plant leaf extracts have been shown not to possess suitable efficacy as a cell culture supplement for keratinocytes or corneal epithelial cells. Particular extracts derived from seeds, such as soybeans, corn, wheat, and rice; and storage organs such as potatoes are explicitly excluded from the set of suitable extracts derived from seed-bearing plants if and only if the extracts are hydrolyzed. As shown in FIGS. 1A and 1B, and discussed above, aloe vera leaf extracts do not possess the ability to support the primary culture and/or cultivation of normal cells.

Substrates for use with the Basal Media and/or the Cell Culture Supplements

Any suitable vessel or cell culture container may be used as a support for cell cultures in the basal media and/or the cell culture supplements. No substrate coating on the support is necessary. Coating the surface of a culture vessel with adhesion-promoting substrata (for example, collagens, fibronectins, RGD-containing polypeptides, gelatins, and the like) however promotes attachment of the cells and thereby enhances the effect of the cell culture media and supplements disclosed herein.

### EXAMPLES

1. Cell Culture Media Supplements

Certain embodiments of the cell culture media supplements comprise simple non-hydrolyzed plant extracts and/or extracts derived from seed-bearing plant material, such as seeds (e.g., rice, wheat, pearl barely, alfalfa, sunflower, poppy, flax, millet, quinoa, pumpkin and sesame, almond, brazil, walnut, pecan, cashew, pine, hazel, filbert, peanuts, lentils, peas, bean, chick pea and soybean). Such embodiments of the cell culture media supplements may be formed by, for example, as follows: Shelled or husked grains, tree nuts, seeds, or legumes were placed in a clean blender and ground for 5 to 10 minutes into a semi-fine powder (the average particle size was approximately less than or equal to 1 mm diameter). A phosphate-buffered saline (PBS, pH 7.4), or other suitable aqueous buffer solution (as known to those persons of ordinary skill in the art), was added to the ground plant at a ratio of 1 part nut, seed or legume, to 2 to 4 parts PBS or other aqueous buffer solution (weight/volume). The mixture was blended for 5 to 10 minutes or until a slurry was formed.

Using a swinging bucket rotor in a centrifuge (IEC HN-II), the slurry was centrifuged at approximately 300×g for 15 minutes. The aqueous phase was removed from the centrifuge and strained through a 700 μm filter to provide a semi-clarified extract. Further, to facilitate formation and removal of cold-insoluble material, certain embodiments of the semi-clarified non-hydrolyzed plant extracts were frozen for at least 30 minutes at or below −20° C., thawed, and then centrifuged at high speed (28,100×g) for 60 minutes at 4° C., using a Sorvall SA-600 rotor. Cold-insoluble material need not be removed from the solutions but may be removed for, e.g., cosmetic reasons. The extract (with or without freeze/thawing and high speed centrifugation steps) was passed through syringe sterilization filters (0.45 μm and 0.2 μm pore size), aliquoted into sterile cryovials and frozen at −20° C. for future use, referred to herein as “Composition 1”.

Further embodiments of the cell culture media supplements comprise non-hydrolyzed plant extracts derived from plant storage organs that include roots, modified roots, tubers, modified stems, rhizomes, corms, runners and bulbs (such as russet potatoes, sweet potatoes, yams). The plant storage organs may be utilized with or without the plant skin. In attempt to avoid inclusion in the extract of undesirable contaminants such as dirt, endotoxins which may be present on the plant skins, the skins may be removed prior to the extraction procedure. Such embodiments of the cell culture media supplements were formed, for example, by utilizing a 1 to 1 ratio (wet weight to volume) of clean skinless plant storage organ to aqueous buffer. As an example, 50 grams of diced russet potato (without the skin) was placed into a clean blender with 50 mls of PBS or other similar aqueous buffer/solution. The clean and skinless russet potato was blended for 5 to 10 minutes until a slurry was formed (having an average particle size of approximately less than or equal to 1 mm). The slurry was centrifuged at approximately 300×g for 15 minutes and the aqueous phase was removed from the centrifuge tube and strained through a 700 μm filter to provide a semi-clarified extract. Alternatively, to facilitate formation and removal of cold-insoluble material, the semi-clarified non-hydrolyzed plant extract was sometimes frozen for at least 30 minutes, at or below −20° C., thawed, and then centrifuged at high speed (28,100×g) for 60 minutes at 4° C., using a Sorvall SA-600 rotor. The extract (with or without freeze/thawing and high speed centrifugation steps) was then passed through sterilization filters (0.45 μm and 0.2 μm pore size), aliquoted into sterile cryovials and frozen at −20° C. for future use, referred to herein as “Composition 2”.

Further embodiments of the cell culture media supplements comprise combined non-hydrolyzed plant extracts derived from either, one or more plant storage organs, and one or more seeds. Such embodiments of the cell culture media supplements were formed by, for example, placing 1.5 parts (by weight) of shelled Virginia peanuts (an example of a seed), and 1 part (by weight) of diced russet potato (an example of a plant storage organ) in a clean blender and grinding for 5 to 10 minutes. To the blender was added 4 parts (by volume) of phosphate-buffered saline solution. The mixture was blended for 5 to 10 additional minutes to form a slurry, and was subsequently centrifuged at approximately 300×g for 15 minutes. The aqueous phase was removed from the centrifuge tube and strained through a 700 μm filter to provide a semi-clarified extract. The semi-clarified extract was frozen and thawed for at least 30 minutes, at or below −20° C. To further suppress the formation of cold-insoluble material, the osmolarity of the semi-clarified extract was increased by adjusting the NaCl concentration to approximately 1.13 M by the addition of solid NaCl. The extract was then stored at 4° C. for 72 hours and subsequently centrifuged at high speed (28,100×g) for 60 minutes at 4° C., using a Sorvall SA-600 rotor. The aqueous supernatant was removed from the centrifuge, avoiding the transfer of any precipitates. The supernatant was sterilized by passing it through 0.45 μm and 0.2 μm pore size filters, aliquoted in 2 ml volumes in cryovials and frozen at −20° C. The resulting extract was added to cell culture media as a 250× concentrated supplement, referred to herein as “Composition 3”

Alternatively, prior to the sterile filtration process for Composition 3, the combined non-hydrolyzed plant extract was further diluted at a ratio of 2 parts unfiltered Composition 3, to 3 parts PBS that included growth factors: EGF (167 ng/ml), IGF-1 (1.67 μg/ml), hydrocortisone (5.0 μM) and PGE-2 (8.3 μM). This plant extract and growth factor containing mixture was sterilized by passing it through 0.45 μm and 0.2 μm pore size filters, and 5 ml volumes were aliquoted into sterile bottles and frozen at −20° C. The aliquots were later thawed for addition to desired basal culture media as a 100× concentrated APF cell culture supplement, referred to herein as “Composition 4”

2. In Vitro Culture of Normal Human Epithelial Cells

Normal human keratinocytes have been isolated and serially-passaged in a highly-defined environment when a basal keratinocyte cell culture medium, EPILIFE® (available from Cascade Biologics of Portland, Oreg.) was supplemented with a combination of bovine serum albumin (BSA) and bovine transferrin (BTf) purified from bovine serum, recombinant human insulin-like growth factor type-1 (IGF-1), hydrocortisone (Hyd), recombinant human epidermal growth factor (EGF) and prostaglandin E-2 (PGE-2). The presently disclosed APF cell culture media supplements utilize the EPILIFE® basal medium; however, the animal products are replaced with the plant extract supplements described herein. Any suitable basal medium may be used as is known to those persons of ordinary skill in the art.

When keratinocytes are grown in a bovine supplement, the bovine components (particularly BSA) are required for adequate plating of the cells onto tissue culture plastic and the proliferation of the cells once they adhere. Disclosed herein is an assay wherein keratinocytes do not adhere and grow unless BSA or other known growth supplements such as BPE are added. For this assay, keratinocyte Target Cells were used, as discussed above. The keratinocytes Target Cells were then thawed and plated into 24-well plates (2500 cells/cm², 2 cm² per well) in M2 Medium as described above for FIG. 1. This assay was used to determine the efficacy of certain of the multiple embodiments of the plant extract cell culture media supplements disclosed herein. Accordingly, certain embodiments of the cell culture media supplements were tested for their ability to suitably replace the activity of BSA/BTf or BPE.

### Example 1

As shown in Table 3 below, human keratinocytes cultured M2 Medium in the absence of animal-derived products (BSA, serum, xenotrophic feeder layer cells, Transferrin, BPE) were induced to adhere, spread and proliferate by the addition of an embodiment of the APF cell culture media supplements disclosed herein.

The results shown in Table 3 demonstrate that certain embodiments of the plant-derived extract supplements act as partial or complete substitutes for animal-derived supplements such as BSA/BTf or BPE to promote the plating and proliferation of neonatal human keratinocytes. As can be seen from the data recited in Table 3, certain embodiments of the disclosed extract supplements from some but not all plant material possess proliferation promoting activity. The data also demonstrate that the disclosed extract supplements from some, but not all plant storage organs possess proliferation activity. Additionally, purified peanut lectin does not have significant growth promoting activity when compared to the disclosed supplement comprising non-hydrolyzed extract of peanuts. The most active extract supplements disclosed herein are those derived from certain seeds (e.g., peanut, sunflower, hazelnut, almond, soybean, corn, chick pea). Moderate growth-promoting activity was observed upon addition of the extracts of other seeds (e.g., pea, rice, wheat), and storage organs (e.g., russet, red, and white potatoes).

The data recited in Table 3 was obtained as follows. Keratinocyte Target Cells were cultured in 24-well cell culture plates in M2 Medium with or without the addition of non-hydrolyzed plant extracts prepared as described for Composition 1 and Composition 2 above without the freezing and thawing steps. The Compositions were added to M2 Medium at concentrations ranging from 1:10,000 to 1:50 final dilutions. Fruit juices (as shown in Table 3) and purified peanut lectin were tested in a similar fashion. After 6-7 days of incubation, plating and proliferation was assessed visually after fixation of cells in 70% isopropanol and staining of cells with 0.1% methylene blue. The activity of each additive was assessed relative to the growth-promoting activity of 0.2% BPE which was given an arbitrary score of 4 on a scale of 0-4. Data is representative of maximal activities attained from the concentration-dependent responses to each additive. Data points were performed in duplicate or triplicate.

TABLE 3
Relative Plating and Proliferation-promoting Activity
of Simple Non-hydrolyzed Plant-derived Extracts
and Juices on Keratinocyte Target Cells

| | Approximate Activity |
|---|---|
| | (Relative to BPE): |
| Supplement Added: | |
| No Addition | 0 |
| Animal (Bovine) Product: | |
| BPE | 4 |
| BSA + Transferrin | 3 |
| BSA | 3 |
| Root Storage Organs: | |
| Onion | 0 |
| Jicama | 0 |
| Red Beet (Harvard) | 0 |
| Carrot | 1 |
| Turnip | 1 |
| Potato (Russet) | 2 |
| Potato (small white) | 2 |
| Potato (small red) | 2 |
| Potato (sweet) | 1 |
| Yam | 1 |
| Grain Extracts: | |
| Rice (brown) | 2 |
| Wheat (kamut) | 2 |
| Pearl Barley | 2 |
| Seed Extracts: | |
| Sunflower | 3 |
| Pumpkin | 1 |
| Flax | 1 |
| Poppy | 1 |
| Alfalfa | 1 |
| Sesame | 1 |
| Corn/Maize (immature) | 1 |
| Corn/Maize (mature-dried, popcorn) | 3 |
| Millet | 2 |
| Quinoa | 2 |
| Tree Nut Extracts: | |
| Hazel/Filbert | 3 |
| Almond | 4 |
| Brazil | 1 |
| Walnut | 1 |
| Pecan | 1 |
| Cashew | 1 |
| Pine | 1 |
| Legume Extracts: | |
| Peanut (dried Raw Spanish) | 4 |
| Peanut (dried Raw Virginia) | 4 |
| Pea (dried green) | 2 |
| Lentil (dried yellow) | 2 |
| Bean (dried small pink) | 2 |
| Soybean | 3 |
| Chick Pea (Garbonzo Bean) | 3 |
| Miscellaneous Extracts: | |
| Banana | 0 |
| Avocado Pit | 0 |
| Fruit Juice: | |
| Cantaloupe | 0 |
| Kiwi | 0 |
| Apple | 0 |
| Plum | 0 |
| Strawberry | 0 |
| Grape | 0 |
| Tomato | 0 |
| Peach | 0 |
| Avocado (extract) | 0 |
| Miscellaneous: | |
| Purified Peanut Lectin | 0.5 |
| (0.5-64 mg/ml; Sigma-Aldrich Chemical) | |

The results illustrated in Table 3 above refer to certain specific embodiments of the cell culture supplements disclosed herein and do not necessarily reflect the results for all embodiments of such supplements including such extracts.

### Example 2

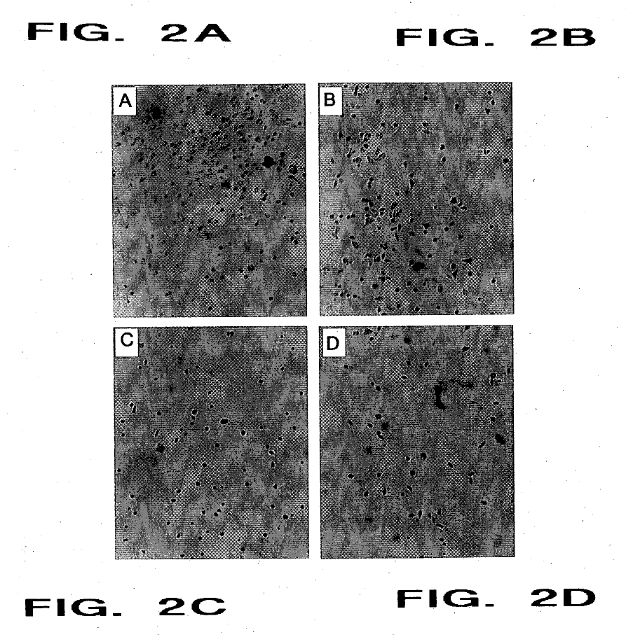

The effect of certain embodiments of the non-hydrolyzed extracts derived from plant storage organs (e.g., russet potatoes) when added to M2 Medium is primarily the promotion of adherence and spreading of human keratinocytes as shown in FIGS. 2A-2D. FIGS. 2A-2D show photomicrographs taken approximately 24 hours after keratinocyte Target Cells were plated in M2 Medium and cultured in the indicated conditions. FIGS. 2A-2D demonstrate that the disclosed embodiments of the extract supplements from plant storage organs (e.g., russet potatoes) facilitate the plating of keratinocyte target cells in M2 Medium in the absence of BPE or other animal-derived components.

The keratinocyte Target Cells shown in FIGS. 2A-2D were added to 24-well cell culture plates in M2 Medium as described above in relation to FIGS. 1A and 1B. Non-hydrolyzed russet potato extract was prepared as described above for Composition 2 with no additional freezing/thawing or high speed centrifugation steps, and added to some of the cultures at a 1:2000 final dilution. All experimental points were performed in duplicate. Shown are keratinocyte Target Cells cultured in M2 Medium only (FIGS. 2A and 2C) or M2 Medium supplemented with russet potato extract (FIGS. 2B and 2D).

### Example 3

Table 4 shows that non-hydrolyzed extracts of soybeans have growth promoting activity on keratinocyte Target Cells while the commercial hydrolyzed soybean extract, HY-SOY, does not. The results suggest that hydrolysis during the preparation of the HY-SOY product destroys any growth promoting ability.

TABLE 4

| Supplement Added to M2 Medium | Cells/cm² (×10⁴), +/−SD |
|---|---|
| No Addition | 1.25, +/−0.10 |
| BPE (0.2%) | 4.83, +/−0.35 |
| HY-SOY (200 mg/l) | 0.30, +/−0.03 |
| Non-hydrolyzed Soy Extract (1:1000) | 3.20, +/−0.39 |

The keratinocyte Target Cells of Table 4 were cultured in T-25 cell culture flasks in M2 Medium only (no addition) or in M2 Medium supplemented as indicated in Table 4. The cell culture medium was changed every 48 hours. The plant-derived HY-SOY (Sigma-Aldrich, St. Louis, Mo. product no.82522) cell culture reagent was dissolved in PBS at a concentration of 20 g/l, sterile-filtered through a 0.22 um syringe filter. Non-hydrolyzed soybean extract was prepared as described for Composition 1 (with freeze/thawing and high speed centrifugation steps). Supplements were added to the culture medium at the final concentration indicated in Table 4. Proliferation of the Target Cells was assessed after 5 days of culture by removal of the cells from the flasks and determination of the cell density by counting with a hemocytometer (Improved Neubauer's Counting Chamber from Erma Inc. of Japan, catalog number 03-202-1)

All experimental points were performed in triplicate and each data point represents the average cell density per T-25 flask, +/− the standard deviation (SD).

### Example 4

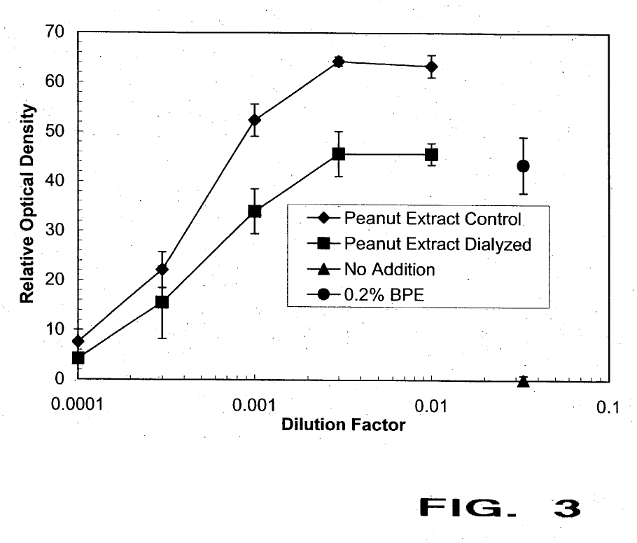

The results shown in FIG. 3 demonstrate that dialysis of peanut extract using dialysis membranes that retain molecules and/or molecular complexes having an average molecular weight equal to or greater than 100,000 Daltons results in a significant retention of the proliferation-promoting activity. As known to those persons of ordinary skill in the art, suitable dialysis membranes are readily available, e.g., a Spectra/Por CE MWCO 100,000; 24 mm may be used. The results suggest that the active proliferation-promoting molecules of peanut extract can be concentrated and possibly purified using size exclusion chromatography, membrane filtration or dialysis or combinations of these.

To obtain such results, keratinocyte Target Cells were cultured and data collected as described for FIGS. 1A and 1B. Simple non-hydrolyzed peanut extract was prepared as described for Composition 1 (with freeze/thawing and high speed centrifugation steps) and substituting HEPES (30 mM)-buffered (pH 7.4) saline for PBS was performed. About 4 mls of the peanut extract was placed into a rinsed dialysis bag (Spectra/Por CE MWCO 100,000; 24 mm) and dialyzed against 3×1 L volumes of HEPES-buffered saline solution over a 72 hour period at 4° C. Control Peanut extract was stored at 4° C. for the duration of the dialysis period. At the end of the dialysis period, the dialyzed peanut extract had increased in volume by 5%. Both control and dialyzed peanut extracts were centrifuged at 400×g for 10 minutes, the supernatants sterile filtered through 0.22 μm syringe filters, and aliquots and stored at −20° C. for future use. The control and dialyzed peanut extracts were thawed and added to M2 Medium at concentrations ranging from 1:10,000 to 1:100 final dilutions, as indicated in FIG. 3. Positive and negative controls are M2 Medium supplemented with 0.2% BPE or M2 Medium without additional supplementation “no addition,” respectively. All experimental points were performed in duplicate.

### Example 5

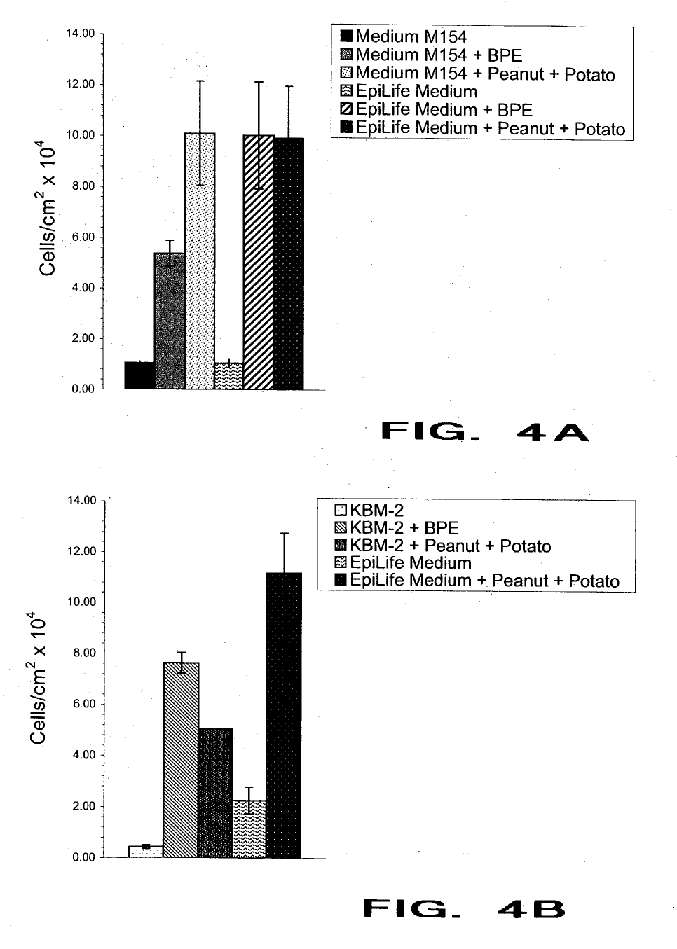

FIGS. 4A and 4B illustrate that similar results may be obtained when the non-hydrolyzed plant extract supplements as disclosed herein in conjunction with basal media other than the EPILIFE® Medium. In this specific embodiment three basal culture media designed to grow human keratinocytes were used: Medium 154 and EPILIFE® Medium (available from Cascade Biologics) and KBM-2 Medium (Cambrex BioScience Walkersville, Inc.). Human keratinocyte Target Cells cultured in the any of these basal media can be induced to adhere, spread and proliferate by the addition of non-hydrolyzed extracts derived from seeds (e.g., sunflower, hazelnut, peanut). Thus, the growth-promoting effects of the disclosed extracts derived from non-hydrolyzed extract supplements, on human keratinocyte cultures are not restricted to cells cultured in any specific basal media.

The keratinocyte Target Cells illustrated in the graphs of FIGS. 4A and 4B were cultured in T-25 cell culture flasks utilizing the cell culture conditions that were similar to that described above in relation to Table 4. Cells were re-suspended in EPILIFE® Medium, Medium 154 (Cascade Biologics) or KBM-2 medium (Cambrex BioScience Walkersville, Inc.; product no. CCM-3107). Each of the listed basal media was supplemented with the M2 Growth Factors. These media were then further supplemented with particular embodiments comprising combined peanut and potato extract supplement (prepared as described for Composition 3), 0.2% BPE, or no additional supplementation, as indicated in FIGS. 4A and 4B.

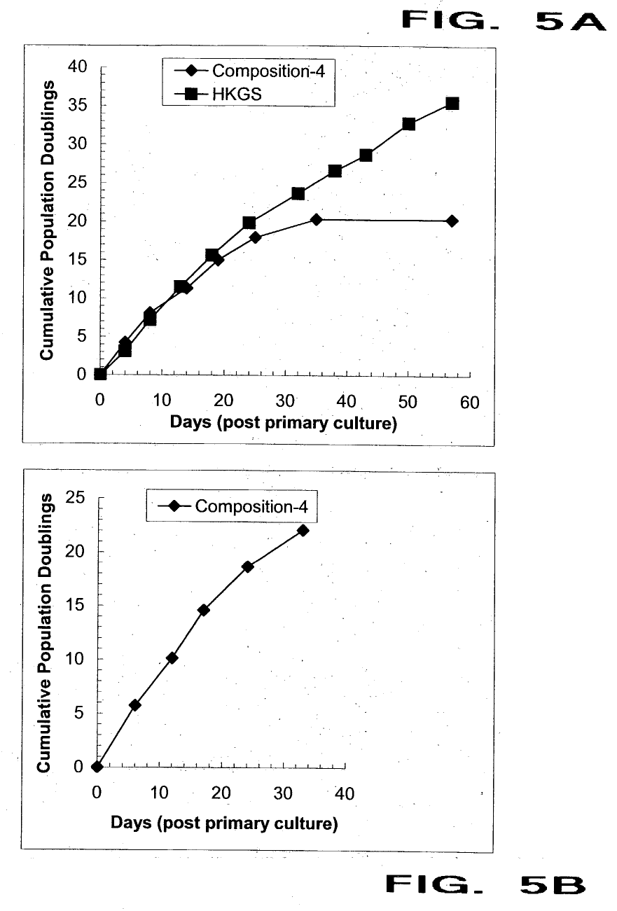

Proliferation of the keratinocyte Target Cells was assessed after either 6 (FIG. 4A) or 5 days (FIG. 4B) of culture by removal of the cells from the flasks and determining the density of the cells with a hemocytometer. All experimental points were performed in triplicate and each data point represents the average cell density +/− the standard deviation (SD).

### Example 6

Primary Culture and Serial Passage of Normal Human Keratinocytes in APF Media.

In the embodiments described in the examples above it has been shown, among other things, that non-hydrolyzed plant-derived extracts can replace animal components for the culture of, for example, keratinocytes that had been previously isolated and grown in media containing animal-derived products. Further, these non-hydrolyzed plant-derived extracts support the efficient primary culture and subsequent serial propagation of, for example, normal human keratinocytes

The results shown in Table 5 demonstrate that non-hydrolyzed plant extracts support the attachment, spreading and proliferation of, for example, keratinocytes in the primary culture and that coating the tissue culture plastic with various matrix materials enhances the number of cells obtained during the culture period. Epidermal cells containing primarily keratinocytes were obtained from normal human foreskin tissue derived from circumcision. The keratinocytes were isolated from the tissue as follows: in an aseptic environment, (a) dermal adipose tissue and excess dermis was surgically removed along with any blood clots; (b) the trimmed skin was cut into pieces of approximately 0.5×1 cm; (c) the tissue pieces were digested for 16-24 hours at 4° C. in dispase (25 units/ml) dissolved in PBS; (d) for each piece of skin the epidermis was physically separated from the dermis; (e) the epidermal pieces were further digested in trypsin (0.025%)/EDTA (0.01%) dissolved in PBS for 30 minutes at 37° C.; (f) the action of the trypsin was stopped by the addition of 2× the volume of 0.0125% trypsin inhibitor (purified from soybean) dissolved in PBS; and (g) the cells were collected by centrifugation and subsequently resuspended in appropriate culture medium. The keratinocytes were seeded into standard tissue culture dishes in EPILIFE Medium that had been supplemented with either the BPE-containing Human Keratinocyte Growth Supplement (available from Cascade Biologics, product no. S-001) or certain embodiments of the plant extracts and supplements disclosed herein.

In particular embodiments the culture surfaces were coated with various matrix materials (as shown in Table 5). After isolation the keratinocytes were suspended in EPILIFE Medium containing an antibiotic/antimycotic solution (PSA; available from Cascade Biologics; product no. R-004), or the same medium supplemented with M2 Growth Factors, or the same medium supplemented as indicated in Table 5 including: an embodiment of the plant-derived supplement prepared as described for Composition 4. Keratinocytes were seeded into tissue culture plastic 6-well plates pretreated with various matrix materials, including: rat type-1 collagen (available from Sigma-Aldrich; product no. C 7661; 15 ug/ml, in 0.1 M Na-Phosphate buffer-pH 8.5); Fibronectin-like Engineered Protein Polymer (FLEP; available from Sigma-Aldrich; product no. F 5147; 20 μg/ml, in phosphate buffered saline, pH 7.4); fetal bovine serum, FBS; or no coating (TC Plastic).

Keratinocytes were seeded at a density of 5,000 viable cells per cm². Cell culture media were replaced every 48 hours. Proliferation of the neonatal keratinocytes in the primary cultures was assessed after 10 days of incubation as described in FIG. 1B. All experimental points were performed in triplicate, and each data point represents the average optical density relative to the untreated control (None), +/− the standard deviation (SD).

TABLE 5

| Supplement Added to | | Relative Optical |
|---|---|---|
| EPILIFE Medium | Matrix Coating | Density, +/−SD |
| M2 Growth Factors | None | 0.0, +/−0.6 |
| HKGS | None | 5.1, +/−0.2 |
| HKGS | Fetal Bovine Serum | 7.6, +/−0.8 |
| HKGS | FLEP | 4.3, +/−0.9 |
| HKGS | Type-1 Collagen (rat) | 8.5, +/−1.4 |
| Composition 4 | None | 14.3, +/−0.9 |
| Composition 4 | FLEP | 43.9, +/−3.2 |
| Composition 4 | Type-1 Collagen (rat) | 51.1, +/−2.2 |

Certain non-hydrolyzed extracts of plants are more effective than others in supporting the growth of keratinocytes in the primary culture. As shown in Table 6 below, extracts from potatoes and peanuts are effective in promoting the growth of, for example, keratinocytes in primary culture. The effects of these two particular embodiments of the disclosed extract supplements may be additive. Prior art plant extract supplements tested were shown not to be effective in supporting the growth of keratinocytes in primary culture, including the Hy-Soy product and Aloe Vera extracts.

As shown in Table 6, epidermal keratinocytes from adult human skin were isolated as described in relation to foreskin tissue in Table 5. The adult keratinocytes were suspended in M2 Medium containing an antibiotic/antimycotic solution (PSA; available from Cascade Biologics; product no. R-004), or the same medium supplemented with various non-hydrolyzed plant extracts as indicated in Table 6. Non-hydrolyzed plant extracts were prepared as described above in relation to the examples set forth for Composition 1. Aloe vera whole leaf powder (available from Terry Laboratories; product no. WL004) and the Hy-Soy hydrolyzed product (available from Sigma-Aldrich; product no. 82522) were dissolved in phosphate-buffered saline solutions (PBS, pH. 7.4) at concentrations of 50 g/L and 40 g/L, respectively, and sterile-filtered through a 0.22 μm syringe filter. Tissue culture 6-well plates were pre-coated with rat type-1 collagen (available from Sigma-Aldrich; product no. C 7661) as described in relation to Table 5 above. Adult keratinocytes were seeded at a density of 13,200 viable cells per cm². Cell culture medium was replaced every 48 hours.

Proliferation of cells in the primary cultures was assessed after 13 days of culture as described in relation to FIG. 1B. All experimental points were performed in triplicate, and each data point represents the average optical density relative to the untreated control (no addition), +/− the standard deviation (SD).

TABLE 6

| | Relative |
|---|---|
| | Optical |
| Supplement Added to M2 Medium | Density, +/−SD |
| None (M2 Medium) | 0.0, +/−0.3 |
| Potato Extract (1:1000) | 3.4, +/−0.4 |
| Sunflower Extract (1:1000) | 0.5, +/−0.7 |
| Sunflower Extract + Potato Extract (1:1000) | 3.2, +/−0.6 |
| Almond Extract (1:650) | 1.1, +/−0.1 |
| Almond Extract + Potato Extract (1:1000) | 2.9, +/−0.2 |
| Hazelnut Extract (1:650) | 0.0, +/−0.2 |
| Hazelnut Extract + Potato Extract (1:1000) | 3.7, +/−0.9 |
| Corn Extract (1:333) | 0.0, +/−0.7 |
| Corn Extract + Potato Extract (1:1000) | 1.9, +/−0.1 |
| Peanut Extract (1:333) | 12.9, +/−1.3 |
| Peanut Extract + Potato Extract (1:1000) | 14.9, +/−1.1 |
| Hy-Soy (200 mg/l) | 0.0, +/−0.1 |
| Hy-Soy (200 mg/l) + Potato Extract (1:1000) | 0.9, +/−0.3 |
| Hy-Soy (400 mg/l) | 0.0, +/−0.4 |
| Hy-Soy (400 mg/l) + Potato Extract (1:1000) | 1.7, +/−0.6 |
| Aloe Vera Extract (400 mg/l) | 0.0, +/−1.2 |
| Aloe Vera Extract (400 mg/l) + Potato Extract (1:1000) | 1.0, +/−0.3 |
| Aloe Vera Extract (500 mg/l) | 0.0, +/−0.2 |
| Aloe Vera Extract (500 mg/l) + Potato Extract (1:1000) | 1.8, +/−0.3 |

Tables 7 and 8 demonstrate that the composition of the basal medium is not essential to the ability of the disclosed non-hydrolyzed plant extract supplements to support the growth of, for example, adult human keratinocytes in the primary culture. Culture media designed to grow human keratinocytes were obtained from three commercial suppliers. EPILIFE® Medium (product no. M-EPI) and Medium 154 (product no. M-154) were obtained from Cascade Biologics; Keratinocyte-Serum Free Medium (Keratinocyte-SFM) was obtained from Invitrogen Corp. of Carlsbad, Calif. (product no. 10724-011); and Keratinocyte Basal Medium-2 (KBM-2) was obtained from Cambrex BioScience Walkersville, Inc. of Walkersville, Md. (product no. CCM-3107). Each of the listed basal media except for Keratinocyte-SFM was supplemented with M2 Growth Factors. Keratinocyte-SFM was supplemented per the manufacturer's directions but without the addition of BPE. All of the tissue culture vessels were coated with type-1 collagen (rat) as set forth above in relation to Table 6. The efficacy of these media were then each tested alone or after further supplementation with certain embodiments of the disclosed supplements, for example, either a combined peanut (1:333 dilution) and potato (1:1000 dilution) extract supplement (prepared as described above for the cell culture supplement Composition 3), or 0.2% BPE. Adult keratinocytes were plated into tissue culture 6-well plates on culture surfaces that had been coated with rat tail type-1 collagen before addition of the cells. Cell culture media were replaced every 48 hours.

Plating and proliferation of the keratinocytes was assessed after 13 days, as described in relation to FIG. 1B. All experimental points were performed in triplicate, and each data point represents the average optical density relative to the untreated control (no addition/TC plastic), +/−the standard deviation (SD).

TABLE 7

| | Supplement Added | Relative Optical |
|---|---|---|
| Basal Medium | (In addition to cytokines and hormones listed above) | Density, +/−SD |
| EpiLife Medium | None | 0.0 +/−0.5 |
| EpiLife Medium | 0.2% BPE | 16.6 +/−1.1 |
| EpiLife Medium | Peanut Extract (1/333) + Potato Extract (1/1000) | 39.2, +/−7.0 |
| Medium 154 | None | 0.0, +/−1.2 |
| Medium 154 | 0.2% BPE | 41.9, +/−3.1 |
| Medium 154 | Peanut Extract (1/333) + Potato Extract (1/1000) | 63.5, +/−0.7 |
| Keratinocyte-SFM | None | 0.0, +/−0.3 |
| Keratinocyte-SFM | 0.2% BPE | 45.0, +/−3.8 |
| Keratinocyte-SFM | Peanut Extract (1/333) + Potato Extract (1/1000) | 14.9, +/−3.2 |

To obtain the results set forth in Table 7, epidermal keratinocytes from adult human skin were isolated as discussed in relation to Table 6 above. The keratinocytes were plated into tissue culture 6-well plates at a seeding density of 18,000 viable cells per cm². Culture surfaces were coated with rat tail type-1 collagen (available from Sigma-Aldrich Chemical; product no. C 7661; 15 ug/ml, 0.1 M Na-Phosphate buffer-pH 8.5) before addition of the media. Cell culture media were replaced every 48 hours. Plating and proliferation of the adult keratinocytes were assessed after 13 days, as described in relation to FIG. 1B. All experimental points were performed in triplicate, and each data point represents the average optical density relative to the untreated control (no addition), +/− the standard deviation (SD).

TABLE 8

| | | Relative |
|---|---|---|
| | Supplement Added (in addition to | Optical |
| Basal Medium | cytokines and hormones listed above) | Density, +/−SD |
| KBM-2 Medium | None | 0.0, +/−1.0 |
| KBM-2 Medium | 0.2% BPE | 2.1, +/−0.4 |
| KBM-2 Medium | Peanut Extract + Potato Extract | 8.0, +/−0.4 |
| EpiLife Medium | Peanut Extract + Potato Extract | 11.6, +/−2.7 |

To obtain the results set forth in Table 8, epidermal keratinocytes from adult human skin were isolated as described for Table 6 above. The keratinocytes were plated into tissue culture 6-well plates at a seeding density of 20,000 viable cells per cm². Culture surfaces were coated with rat tail type-1 collagen (available from Sigma-Aldrich Chemical; product no. C 7661; 15 ug/ml, 0.1 M Na-Phosphate buffer-pH 8.5) before addition of the media. Cell culture media were replaced every 48 hours. Plating and proliferation of the adult keratinocytes were assessed after 13 days, as described in relation to FIG. 1B. All experimental points were performed in triplicate, and each data point represents the average optical density relative to the untreated control (no addition), +/− the standard deviation (SD).

FIGS. 5A and 5B demonstrate that keratinocytes that have been grown in the primary culture using particular embodiments of the disclosed plant extracts can be serially propagated in media with the same supplements. For FIG. 5A, the cells were propagated in the primary culture using tissue culture plastic ware that had been coated with rat tail type-1 collagen. For FIG. 5B, the cells were isolated and grown in the primary culture in animal product-free conditions on tissue culture plastic ware that had not been coated. For both examples, after the primary culture the cells were plated and grown in animal product-free conditions on standard tissue culture plastic ware without coating.

To obtain the results shown in FIG. 5A, epidermal keratinocytes from adult human skin were isolated as described in relation to Table 6 above. Adult keratinocytes were seeded into cell culture T-25 flasks that were coated with rat tail type-1 collagen as described for Table 8 at a density of 20,000 viable cells/cm². Media used were: EPILIFE Medium containing an antibiotic/antimycotic solution (PSA; available from Cascade Biologics; product no. R-004) supplemented with an embodiment of the disclosed supplements, specifically Composition 4, or the same basal medium supplemented with BPE-containing Human Keratinocyte Growth Supplement, HKGS (available from Cascade Biologics; product S-001), and PSA. Cell culture media were replenished every 48 hours.

The cells in the primary cultures were removed from the flasks after 9 days of incubation and the cell density determined by counting the cells with a hemocytometer. The average cell density for triplicate flasks of adult keratinocytes cultured in an embodiment of the disclosed supplements was 2.18×10⁴ cells/cm² and for the HKGS supplemented medium 2.00×10⁴ cells/cm².

The adult keratinocytes were subsequently resuspended in their corresponding media and seeded at a density of 2.5×10³ cells/cm² in the same type of culture vessel (but without type-1 rat collagen coating) in triplicate. The culture media were replenished every 48 hours. When the cell density reached approximately 80% confluency, cultures in each condition were harvested, cell numbers determined and new subcultures established. The number of population doublings at each passage was calculated and shown in the FIG. (5A). Each data point shown represents the average cumulative population doubling, +/− the standard deviation (SD).

To obtain the results shown in FIG. 5B, epidermal keratinocytes from neonatal human foreskin were isolated as previously described in Table 5 (as set forth above) except that no animal-derived products were used (i.e., recombinant trypsin was used instead of the described trypsin in the trypsin/EDTA solution.) The neonatal keratinocytes were suspended in EPILIFE Medium containing an antibiotic/antimycotic solution (PSA; available from Cascade Biologics; product no. R-004,) supplemented with a particular embodiment of the disclosed extracts and supplements, specifically Compostion 4. The cells were seeded into standard tissue culture plastic ware at a density of 10,000 viable cells/cm². The cell culture medium was replenished every 48 hours.

The cells in the primary cultures were harvested after 11 days of incubation and the cell density determined by counting the cells with a hemocytometer. The cell density upon harvesting the cells was 5.9×10⁴ cells/cm². The neonatal keratinocytes were subsequently collected by centrifugation, resuspended and reseeded in the same medium. The cultures were serially passaged when the cultures became approximately 80% confluent using recombinant trypsin/EDTA solution (available from Cascade Biologics, product number R-009) and defined trypsin inhibitor solution (available from Cascade Biologics; product no. R-007) at each passage. Each data point represents the average cumulative population doubling at each passage, +/− the standard deviation (SD).

### Example 7

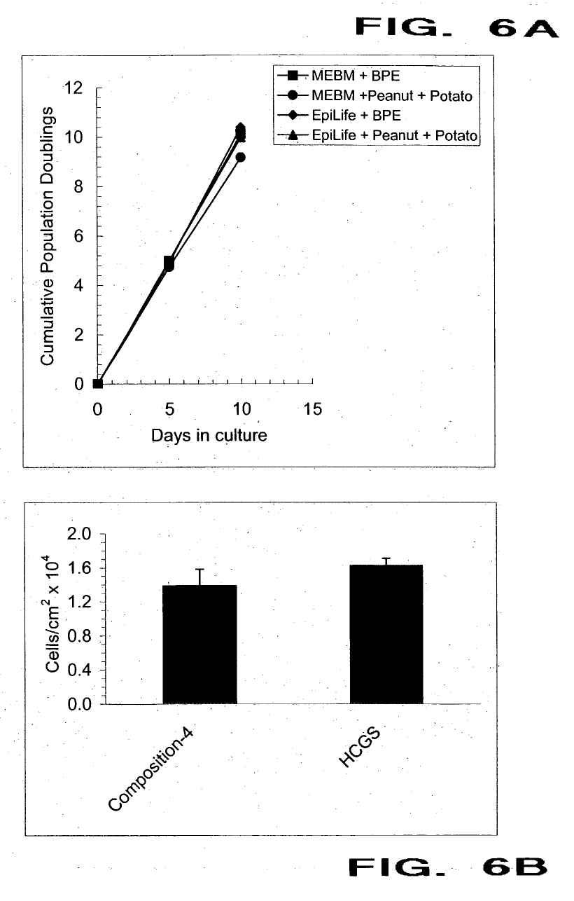

As shown in FIGS. 6A and 6B, an embodiment of the disclosed supplements comprising a combined peanut and potato extract supplement supports the plating and proliferation of two other normal human epithelial cell types: mammary epithelial cells and corneal epithelial cells. For human mammary epithelial cells (HMEC) it is shown that the effect is not dependent on the basal medium that is used (FIG. 6A).

Specifically, to obtain the results shown in FIG. 6A, eighth passage normal human mammary epithelial cells (HMEC; available from Cambrex BioScience Walkersville, Inc.; product no. CC-2551) that had been grown according to the manufacturer's recommendation were harvested from stock cultures and resuspended in experimental medium. The cells were seeded at 2,500 cells/cm² in cell culture T-25 flasks. Experimental media were EPILIFE® Medium (available from Cascade Biologics; product no. M-EPI-500) supplemented with hydrocortisone (0.18 ug/ml), IGF-1 (10 ng/ml) and EGF (10 ng/ml); or Mammary Epithelial Basal Medium (MEBM) (available from Cambrex BioScience Walkersville, Inc.; product no. CC-3151) supplemented with EGF (10 ng/ml), bovine insulin (5 ug/ml) and hydrocortisone (0.5 ug/ml). These media were further supplemented with either the combined peanut and potato extract supplement (prepared as described for Composition 3) or 0.2% BPE, as indicated in FIG. 6A. Proliferation of the HMEC cultures was assessed at the indicated times by removing the cells from the flasks and determining the cell number with a hemocytometer.

Cells were then collected by centrifugation and re-suspended and re-seeded in their corresponding experimental cell culture conditions. All experimental points were performed in triplicate and each data point represents the average cumulative population doublings +/− the standard deviation (SD).

To obtain the results shown in FIG. 6B, cryopreserved third culture normal human corneal epithelial cells (HCEC-3; available from Cascade Biologics; product no. C-019-SC) were re-suspended in experimental medium and seeded at 2,500 cells/cm² in cell culture T-25 flasks. Experimental media were EPILIFE Medium supplemented with an embodiment of the disclosed supplements comprising a combined peanut extract and potato extract that included growth factors (prepared as described for Composition 4), or EPILIFE® Medium supplemented with Human Corneal Growth Supplement (HCGS) (available from Cascade Biologics; product no. S-009-5), a 0.2% BPE-containing growth supplement for HCEC-3 cells. Proliferation of the HCEC cultures was assessed at the described for FIG. 6A at day 8 (Composition 4) and day 7 (HCGS) after seeding. All experimental points were performed in triplicate and each data point represents the average cell density +/− the standard deviation (SD).

### Example 8

The results recited in Table 9 below illustrate the concentration dependent growth response of keratinocyte Target Cells to several non-hydrolyzed extracts of plant material as accessed by scanning densitometry. These results demonstrate that extract supplements from specific plant seeds (e.g., sunflower, corn, hazelnut, almond, peanut), and a plant storage organ (e.g., russet potato) possess significant growth promoting activity.

Corn and potato extract supplements may be of particular interest as they are potentially less allergenic than extracts derived from certain other plants and may represent supplements especially suitable for the culture of cells and/or the production of cell-derived products destined for treatment of humans.

TABLE 9

| | |
|---|---|
| Supplement Added to M2 Medium | Relative Optical Density, +/−SD |

| | |
|---|---|
| No Addition | 0.0, +/−0.2 |
| 0.2% BPE | 36.8, +/−0.8 |
| Peanut Extract (1:3330) | 14.1, +/−1.7 |
| Peanut Extract (1:1000) | 39.0, +/−2.8 |
| Peanut Extract (1:333) | 55.4, +/−0.1 |
| Peanut Extract (1:100) | 21.2, +/−3.9 |
| Corn Extract (1:1000) | 8.6, +/−0.6 |
| Corn Extract (1:333) | 15.6, +/−4.1 |
| Corn Extract (1:100) | 0.0, +/−1.9 |
| Corn Extract (1:50) | 0.0, +/−2.0 |
| Almond Extract (1:3330) | 13.1, +/−1.5 |
| Almond Extract (1:1000) | 26.6, +/−2.8 |
| Almond Extract (1:333) | 43.8, +/−3.6 |
| Almond Extract (1:100) | 18.1, +/−4.8 |
| Sunflower Extract (1:3330) | 10.1, +/−0.9 |
| Sunflower Extract (1:1000) | 11.0, +/−3.3 |
| Sunflower Extract (1:333) | 0.0, +/−0.3 |
| Sunflower Extract (1:100) | 0.0, +/−0.7 |
| Hazelnut Extract (1:3330) | 7.0, +/−1.1 |
| Hazelnut Extract (1:1000) | 15.1, +/−3.4 |
| Hazelnut Extract (1:333) | 16.3, +/−2.4 |
| Hazelnut Extract (1:100) | 5.4, +/−0.5 |
| Russet Potato Extract (1:3330) | 10.7, +/−0.7 |
| Russet Potato Extract (1:1000) | 12.0, +/−0.3 |
| Russet Potato Extract (1:333) | 16.6, +/−0.9 |
| Russet Potato Extract (1:100) | 10.0, +/−0.5 |

The keratinocyte Target Cells of Table 9 were seeded in 24-well cell culture plates in M2 Medium and data collected as described above for FIGS. 1A and 1B. Simple non-hydrolyzed plant-derived extracts were prepared as described in relation to Compositions 1 and 2 and added to the medium at concentrations ranging from 1:3330 to 1:50 final dilutions, as indicated in Table 9. Positive and negative controls were M2 Medium supplemented with 0.2% BPE or M2 Medium with no additional supplementation, respectively. Proliferation of the keratinocyte Target Cells was assessed after 6 days of culture as described above for FIG. 1B. All experimental points were performed in triplicate. Each data point represents the average optical density relative to the untreated control (no addition), +/−the standard deviation (SD).

### Example 9

The results recited in Table 10 illustrate that non-hydrolyzed extract supplements of specific seeds such as tree nuts (e.g., hazelnut) and legumes (e.g., peanut) display proliferation-promoting activity on keratinocyte Target Cells that can be enhanced by the addition of a non-hydrolyzed extract of plant storage organ (russet potato).

TABLE 10

| | Relative Optical |
|---|---|
| Supplement Added to M2 Medium | Density, +/−SD |
| No Addition | 0.0 +/−1.4 |
| Peanut Extract (1:333) | 24.3 +/−2.9 |
| Peanut Extract (1:333) + Potato Extract (1:2000) | 57.1 +/−4.7 |
| Hazelnut Extract (1:333) | 15.6 +/−8.7 |
| Hazelnut Extract (1:333) + Potato Extract (1:2000) | 46.3 +/−7.8 |
| BSA (30 ug/ml) + Transferrin (5 ug/ml) | 5.7 +/−2.5 |
| BSA (30 ug/ml) + Transferrin (5 ug/ml) + Potato | 5.9 +/−1.3 |
| (1:2000) | |

The keratinocyte Target Cells (Table 10) were seeded in 24-well cell culture plates in M2 Medium as described for FIGS. 1A and 1B, above. Non-hydrolyzed extract supplements of plants were prepared as described above in relation to Compositions 1 and 2 with no additional freezing/thawing or high speed centrifugation step, and added to the M2 Medium at 1:333 final dilution (Peanut and Hazelnut extracts) or 1:2000 final dilution (Russet Potato extract), as indicated in Table 10. Positive and negative controls were M2 Medium supplemented with BSA (30 ug/ml) and Bovine Transferrin (5 ug/ml) or M2 Medium without additional supplementation (No Addition), respectively. Proliferation of the keratinocyte Target Cells was assessed after 6 days of culture as described above for FIG. 1B. All experimental points were performed in duplicate and each data point represents the average optical density relative to the untreated control (no addition), +/− the standard deviation (SD).

The specific embodiments and examples disclosed herein demonstrate that non-hydrolyzed plant extracts and/or extracts derived from portions of seed-bearing plants, such as seeds and/or root storage organs can efficiently replace the adherence and proliferation-promoting activities of animal-derived products and allow for both the efficient primary isolation (keratinocytes) and serial propagation of normal human epithelial cells (keratinocytes, mammary epithelial cells, corneal epithelial cells) under APF culture conditions.

The disclosed plant-derived extracts were shown to be effective as cell culture supplements to a chemically-defined cell culture medium containing growth factors and hormones, to support the primary culture (keratinocytes) or propagation (of, for example, keratinocytes, mammary epithelial cells, corneal epithelial cells) of normal animal cells in an APF cell culture environment.

The use of these plant-derived supplements for primary culture and/or cultivation of cells may be further enhanced by pre-coating the surface of the culture dish with adhesion-promoting substrata (for example, collagens, fibronectins, RGD-containing polypeptides, gelatin or various other extracellular matrix proteins).

While this disclosure has been illustrated using certain specific embodiments, it will be understood that further modifications may be made and this application covers any variations, uses, or adaptations of the disclosed embodiments following, in general, the principles of the invention and including such departures from the present disclosure as come within known or customary practice within the art to which the invention pertains and as may be applied to the features herein and as follows in scope of the appended claims.

1. A cell culture medium supplement comprising:
an extract derived from a portion of a seed-bearing plant;
wherein the culture medium supplement supports the cultivation of cells from animal tissue, in vitro.

2. The supplement of claim 1, wherein the culture medium supplement further supports the proliferation of cell cultures from animal tissue, in vitro.

3. The supplement of claim 1, wherein the culture medium supplement further supports the differentiation, primary culture and/or propagation of cell cultures from animal tissue, in vitro.

4. The supplement of claim 1, wherein the extract is non-hydrolyzed.

5. The supplement of claim 1, wherein the extract is derived from a storage organ.

6. The supplement of claim 1, wherein the extract is derived from a seed.

7. The supplement of claim 1, wherein the extract is derived from at least one seed comprising a sunflower, pumpkin, alfalfa, poppy, millet, wheat, barely, rice, corn, quinoa, flax or sesame seed.

8. The supplement of claim 1, wherein the extract is derived from at least one seed comprising an almond, hazelnut, brazil nut, cashew, pine nut, pecan, walnut, or filbert nut.

9. The supplement of claim 1, wherein the extract is derived from at least one seed comprising a bean, peanut, lentil, soybean, pea, or chick pea.

10. The supplement of claim 1, wherein the extract is derived from a peanut.

11. The supplement of claim 1, wherein the extract is derived from a mixture of potato and peanut extracts.

12. The supplement of claim 1, wherein the extract is derived from a mixture of potato and almond extracts.

13. The supplement of claim 1, wherein the extract is derived from a mixture of potato and hazelnut extracts.

14. The supplement of claim 1, wherein the culture medium supports the cultivation of epithelial cells.

15. The supplement of claim 1, wherein the culture medium supplement supports the cultivation of mammary epithelial, corneal epithelial, and/or keratinocyte cell cultures.

16. The supplement of claim 1, wherein the extract comprises molecules and/or molecular complexes having an average mass of about 100,000 Daltons or greater.

17. A cell culture medium comprising:
a supplement comprising a non-hydrolyzed plant extract; and
a basal medium;
wherein the culture medium supports the primary culture and/or cultivation of cell cultures from animal tissue, ex vivo.

18. The cell culture medium of claim 17, further comprising serums, tissue extracts, growth factors, hormones, or cytokines.

19. The cell culture medium of claim 17, wherein the extract is derived from a peanut.

20. The cell culture medium of claim 17, wherein the extract is derived from a storage organ.

21. The cell culture medium of claim 17, wherein the extract is derived from a seed.

22. The cell culture medium of claim 17, wherein the culture medium supplement supports the cultivation of mammary epithelial, corneal epithelial, and/or keratinocyte cell cultures.

23. A cell culture medium comprising:
a supplement comprising an extract derived from at least a portion of a seed-bearing plant; and
a basal medium;
wherein the culture medium supports the primary culture and/or cultivation of cell cultures from animal tissue, ex vivo.

24. The cell culture medium of claim 23, wherein the extract is derived from a storage organ and/or a seed.

25. The cell culture medium of claim 23, wherein the extract is derived from a seed.

26. The cell culture medium of claim 23, wherein the culture medium supplement supports the cultivation of mammary epithelial, corneal epithelial, and/or keratinocyte cell cultures.

27. A method of cultivating an animal cell comprising:
(a) contacting the animal cell culture with the culture medium supplement of claim 1; and
(b) cultivating the animal cell culture under conditions suitable to support cultivation of the animal cell culture.

28. The method of claim 27, wherein the animal cell culture comprises mammary epithelial, corneal epithelial, and/or keratinocyte cell cultures.

29. A method of cultivating an animal cell comprising:
(a) contacting the animal cell culture with the culture medium supplement of claim 17; and
(b) cultivating the animal cell culture under conditions suitable to support cultivation of the animal cell culture.

30. The method of claim 29, further comprising the step of contacting the animal cell culture with the culture medium supplement of claim 17 in a culture vessel coated with adhesion-promoting substrata.

31. The method of claim 30, wherein the adhesion-promoting substrata comprises FLEP and/or collagen.

32. A method of producing a cell culture medium having no animal-derived components, comprising combining a basal cell culture medium that comprises no animal-derived components with a non-hydrolyzed plant extract, wherein the basal cell culture medium and non-hydrolyzed plant extract are present in an amount that supports the primary isolation and cultivation of cell cultures from animal tissue, in vitro.

33. A kit for cultivation of cells from animal tissue, in vitro, comprising:
a supplement comprising a non-hydrolyzed plant extract;
animal tissue cells; and
a basal medium;
wherein the supplement and basal medium have no animal-derived components.

34. The kit of claim 33, wherein the cells from animal tissue comprise mammary epithelial, corneal epithelial, and/or keratinocyte cells.

35. The kit of claim 33, wherein the non-hydrolyzed plant extract is derived from a seed and/or a storage organ.

36. A cell culture medium supplement comprising composition 3.

37. A cell culture medium supplement comprising composition 4.
